(19) United States
(12) Patent Application Publication
(10) Pub. No.: US 2006/0142615 A1
(43) Pub. Date: Jun. 29, 2006

(54) Process for preparing ethanolamine with improved colour

(76) Inventors: Daniel Brun-Buisson, Istres (FR); Mathias Mauvezin, Martigues (FR); Florence Ponsi, Trets (FR)

Correspondence Address: NIXON & VANDERHYE, PC, 901 NORTH GLEBE ROAD, 11TH FLOOR, ARLINGTON, VA, 22203, US

(21) Appl. No.: 10/559,707

(22) PCT Filed: May 12, 2004
(86) PCT No.: PCT/GB04/02023

§ 371 Date: Dec. 7, 2005

(30) Foreign Application Priority Data
Jun. 23, 2003 (FR) 0307542

(51) Int. Cl. C07C 213/02

(52) U.S. Cl. 564/503

(57) ABSTRACT

The present invention relates to a process for preparing an ethanolamine having an improved colour quality. The process comprises contacting ethanolamine with an activated carbon free of one or more metals chosen from Re, Ru, Rh, Pd, Os, Ir, Pt and Ag. The contacting can be carried out at a temperature of from 10 to 200° C., and during a period sufficient to reduce the colour of the ethanolamine, in particular a period such that the colour index (as measured according to the ASTM standard D 1209) of the ethanolamine becomes equal to or less than 50 or 40 Pt/Co. The contacting can be carried out during or after the stage of preparation of the ethanolamine, preferably during or after the stage of purification of the ethanolamine. The invention also relates to a process for manufacturing a triethanolamine (TEA) having an improved colour quality, comprising a stage (i) of synthesis of the TEA by contacting ammonia with ethylene oxide in aqueous medium, a stage (ii) of separation of a crude TEA from the aqueous medium and a stage (iii) of purification of the TEA by distillation. The process in addition comprises contacting the crude or purified TEA with an activated carbon free of one or more metals chosen from Re, Ru, Rh, Pd, Os, Ir, Pt and Ag, after the separation stage (ii), or during and after the purification stage (iii). The advantage of the claimed invention is provide an ethanolamine which has an improved colour quality far more resistant over time, and which is obtained in the absence of any additive or metal catalyst known to contaminate the ethanolamine.

The present invention relates to a process for discolouring an ethanolamine, together with a process for manufacturing a triethanolamine (TEA) having an improved colour quality.

Ethanolamines are used in the preparation of numerous products such as dispersing agents, emulsifying agents, soaps, detergents and shampoos, and in particular in the cosmetics industry and the pharmaceutical industry.

It is known that a pure ethanolamine, obtained more particularly by distillation of a crude ethanolamine which has itself been prepared for example by reacting ammonia with ethylene oxide, can be immediately coloured, or conversely can be immediately colourless, but can in this case generally colour rapidly during its storage. The phenomenon of the colouring of ethanolamines is in particular described in “SRI International, Process Economics Program Report no. 193” of January 1991, pp. 6-9 and 6-10. It seems, however, that the phenomenon of the colouring of ethanolamines is difficult to explain and control, and that the various solutions proposed are generally not very satisfactory. The proposed solutions generally consist in using special materials such as stainless steels in the manufacture and storage of the ethanolamines, or else in using an additive that reduces or suppresses the colouring. However, in said last case the presence of an additive in the ethanolamines can affect the degree of purity of the latter and give rise to other toxicological problems linked to the use of the additive.

American patent U.S. Pat. No. 3,819,710 discloses a process for improving the colour and the stability of the colour of ethanolamines. The process comprises a hydrogenation of crude ethanolamines obtained beforehand by reacting ammonia with ethylene oxide. The hydrogenation is carried out in the presence of hydrogen and a metallic hydrogenation catalyst chosen from catalysts based on Raney nickel, platinum, palladium or ruthenium, at a relatively high temperature of from 60 to 130° C.

American patent U.S. Pat. No. 2,744,938 discloses a process for removing colour impurities from alkylphenol comprising a hydrogenation treatment of the alkylphenol. More particularly, the process comprises contacting an alcohol solution of the alkylphenol with an activared carbon at a temperature of from about 40° to about 150° C. in the presence of hydrogen maintained in the contacting zone at a superatmospheric pressure sufficient to maintain said alcohol substantially in liquid phase.

American patent U.S. Pat. No. 6,291,715 discloses a process for preparing alkanolamines having an improved colour quality. The process comprises a hydrogenation of the alkanolamines that is carried out in the presence of hydrogen and a supported metal catalyst, at a relatively high temperature of from 70 to 160° C. The catalyst is chosen from heterogeneous hydrogenation catalysts comprising one or more metals chosen from rhenium, ruthenium, rhodium, palladium, osmium, iridium, platinum and silver, and supported on a solid support chosen from activated carbon, alpha-alumina, zirconium dioxide and titanium dioxide.

It turns out that one or the other of the processes described previously has the drawback of employing a specific, laborious and costly stage, so-called hydrogenation in the presence of hydrogen, which is carried out at a relatively high temperature and in the presence of a hydrogenation catalyst whose metallic elements can reappear in trace form in the alkanolamines. In addition, American patent U.S. Pat. No. 6,291,715 does not show any advantage in using a support other than another of those proposed for the hydrogenation catalyst.

American patent U.S. Pat. No. 3,723,529 discloses a process for decolorizing “polyethylene polyamines”, such as triethylenetetramine, and higher homologues, such as tetraethylenepentamine, by treatment with active carbon at elevated temperatures followed by distillation. Furthermore, it was not suggested that such a process known for decolorizing “polyethylene polyamines” also was useful with similar effects for ethanolamines. On the contrary, there are some literatures which tend to show the opposite.

An extremely simple and inexpensive process that makes it possible to discolour an ethanolamine during or after its manufacture has been found. The process has the advantage of not contacting an ethanolamine with an additive or a catalyst capable of allowing metallic elements dangerous to human health and the environment to escape into the ethanolamine. It has been found, surprisingly, that an ethanolamine, more particularly a triethanolamine (TEA), treated in this way according to the invention generally becomes colourless or, at any rate, exhibits a colour quality that is markedly improved and that is far more resistant over time.

The present invention relates first of all to a process for preparing an ethanolamine having an improved colour quality, characterised in that it comprises a contacting of an ethanolamine with an activated carbon free of one or more metals chosen from rhenium, ruthenium, rhodium, palladium, osmium, iridium, platinum and silver.

By “ethanolamine” is generally understood an ethanolamine or a mixture of two or more ethanolamines chosen in particular from monoethanolamine (MEA), diethanolamine (DEA) and preferably TEA. The process of the present invention is well suited to one or more ethanolamines, preferably TEA, in particular when it (they) is(are) prepared in a synthesis stage by reacting ethylene oxide with ammonia, preferably in aqueous medium, for example according to one of the synthesis processes described in “SRI International, Process Economics Program Report no. 193” of January 1991, pp. 6-1 to 6-9. The synthesis stage is generally carried out by contacting ethylene oxide with ammonia, for example in a molar ratio between ammonia and ethylene oxide of from 0.5/1 to 40/1, preferably from 1/1 to 10/1, in particular from 1.5/1 to 6/1. The synthesis stage takes place, preferably, in aqueous medium, so that the weight ratio between ammonia and water can be from 0.5/1 to 1/1. The synthesis stage is generally carried out at a temperature of from 0 to 150° C., preferably from 20 to 100° C., in particular from 40 to 80° C., and under an absolute pressure that can be from 0.1 to 15 MPa, preferably from 0.2 to 5 MPa, in particular from 0.2 to 2 MPa. The synthesis stage is preferably carried out in aqueous medium and continuously. It generally leads to the formation of a so-called “crude” TEA comprising TEA and one or more other ethanolamines produced during the reaction, in particular MEA, DEA and optionally ethoxylated triethanolamines (TEAE), also called triethanolamine glycol ethers, in a mixture with the aqueous medium and optionally with one of the two agents in excess or not having reacted, such as ammonia. The process for preparing the TEA can then comprise one or more stages for first of all separating the crude TEA from the aqueous medium and optionally from one of the two reagents in excess or not having reacted such as ammonia, preferably by one or more distillations more particularly in series, then for purifying the TEA and in particular for isolating a so-called “purified” TEA from the crude TEA, in particular by separating the TEA from the other ethanolamines produced, such as those mentioned above, preferably by one or more distillations more particularly in series. The stages of separation and purification of the TEA can with advantage be carried out continuously. In a continuous preparation process, the water and any excess of one of the reagents, in particular ammonia, are generally separated continuously from the crude TEA and are with advantage returned continuously into the TEA synthesis stage.

The process of the present invention is particularly well suited to one of the TEAs prepared according to one of the processes mentioned above, in particular a crude TEA or, preferably, a purified TEA such as that described above, and which is in the form of a coloured TEA during or after its preparation. There is meant generally by a “coloured ethanolamine or TEA” an ethanolamine or a TEA having a colour index of more than 40 Pt/Co, preferably more than 50 Pt/Co according to ASTM standard D 1209. Furthermore, there is generally meant by “purified TEA” a TEA having a content by weight of TEA equal to or more than 85%, preferably equal to or more than 90%, more particularly equal to or more than 99%, and being able to contain as main purity generally DEA in a content by weight of less than 15%, preferably less than 10%, more particularly less than 1% or even less than 0.2%.

The process of the invention is suited more generally to an ethanolamine having initially, prior to its contacting with the activated carbon, a colour index (according to ASTM standard D1209) of more than 40 Pt/Co, preferably more than 50 Pt/Co, and optionally a content by weight of metal, more particularly of iron, equal to or more than 6 parts by weight per million (ppm), preferably equal to or more than 8 or 10 ppm. The method of analysis for measuring the iron content in an ethanolamine such as TEA can be more particularly carried out by plasma emission spectrometry (ICP-AES) using a commercial reference spectrometer “Iris Advantage”® sold by Thermo Jarrel Ash (France), at a wavelength of 259.94 nm.

The process of the present invention is in particular characterised in that it comprises a contacting of an ethanolamine with an activated carbon which generally represents a substantially amorphous form of carbon. The activated carbon has in particular a large specific surface area, for example a specific surface area (according to the BET N₂ method, in particular as described by Brunauer, Emmett and Teller in J. Am. Chem. Soc., 60, 309 (1938)) of from 500 to 5000 m²/g, preferably from 500 to 2500 m²/g, in particular from 700 to 2000 m²/g. The activated carbon generally has a highly porous structure and can have a total pore volume (according to the DIN 66134 method) of from 0.05 to 2.5 cm³/g, preferably from 0.2 to 2.5 cm³/g, in particular from 0.5 to 1.5 cm³/g. The activated carbon can be in the form of powder, granules or shaped products such as rings, cylindrical pellets or balls, and have a mean particle size (by weight) of from 0.1 to 5 mm, preferably from 0.2 to 3 mm, more particularly from 0.5 to 2 mm. It can have a bulk density of from 0.2 to 0.8 g/cm³, preferably from 0.3 to 0.6 g/cm³. It can have an abrasion number/index (according to the ASTM D 4058-87 method) of from 30 to 100, preferably from 50 to 90, and a hardness index/number (or crushing strength) of from 50 to 100 N, preferably from 90 to 98 N (in particular according to the method described by “Ullmann's Encyclopedia of Industrial Chemistry, 5th completely revised Ed., Vol. A 5, Chapter 6.3, p. 356, 2nd paragraph”, in particular when the activated carbon is in granule form).

The active carbon used according to the invention is in particular free of one or more metals chosen from rhenium, ruthenium, rhodium, palladium, osmium, iridium, platinum and silver. By “free of one or more metals” is meant generally an active carbon containing less than 0.05%, preferably less than 0.01%, in particular less than 0.005% by weight of one or more of said metals, calculated as metal in the zero oxidation state. The active carbon used does not support a catalyst, more particularly a metal catalyst, or in particular a hydrogenation catalyst. Preferably, it is substantially free of any metal, in particular one deposited on it. By “free of any metal” is meant generally an activated carbon containing less than 0.05%, preferably less than 0.01%, in particular less than 0.005% by weight of one or more metals.

The activated carbon is generally prepared by the calcination of organic materials, in particular ones rich in carbon such as coal, lignite, wood, nutshells, peat, pitch, coal tar and coke. It can be prepared by a thermal activation process comprising, for example, a stage of the thermal decomposition or carbonisation of the raw material, then a stage of the controlled gasification or activation of the crude carbonisation product. The activated carbon can also be prepared by a chemical activation process comprising, for example, a stage of impregnation of the raw material, more particularly wood, by a chemical activation agent such as phosphoric acid or potash, or a Lewis acid such as aluminium chloride, ferric chloride or zinc chloride, then a stage of heating of the mixture thus obtained to a temperature of from 450 to 700° C. and a drying stage.

In the process of the present invention, the contacting of the ethanolamine with the active carbon can be carried out at a temperature chosen in a range of from 10 to 200° C., preferably from 15 to 100° C., notably from 20 to 80 C, in particular from 25 to 70° C. and more particularly from 30 to 60° C. It can be carried out under any atmosphere or an inert atmosphere, such as air or nitrogen, preferably a dehydrated atmosphere, in particular an atmosphere free of hydrogen, and more particularly under an absolute pressure of from 10² Pa to 2 MPa, preferably from 0.1 to 0.3 MPa.

The contacting can be carried out in various ways, for example by adding the activated carbon to the ethanolamine, or preferably by adding the ethanolamine to the activated carbon. It is possible in particular to place the activated carbon is suspension in the ethanolamine and then to preferably agitate the suspension thus created. It is possible also to pass the ethanolamine through a fixed activated carbon bed, preferably according to a descending stream. The contacting can be carried out discontinuously or preferably continuously.

The contacting of the ethanolamine with the activated carbon can be carried for a period sufficient to reduce the colour of the ethanolamine, preferably for a period such that the ethanolamine become colourless, or more particularly a period such that the colour index (according to the ASTM standard D 1209) of the ethanolamine becomes equal to or less than 50 or 40 Pt/Co, preferably equal to or less than 30 Pt/Co, more particularly equal to or less than 25 Pt/Co. In general, the mean residence time of the ethanolamine contacted with the activated carbon depends on the temperature at which the contacting is carried out, and on the initial colour index of the ethanolamine, prior to the contacting with the activated carbon. The mean residence time can be chosen from a range of from 10 minutes to 18 hours, preferably from 30 minutes to 12 hours, in particular from 1 to 8 hours.

The activated carbon thus contacted with the ethanolamine can retain progressively compounds contained in the ethanolamine, for example organic compounds or metallic compounds, in particular iron. It is considered that when, in the process of the invention, the initial mass of the activated carbon has increased by about 20 wt % through the accumulation of retained compounds, it is preferable to then subject the activated carbon to a regeneration treatment, for example a treatment comprising a stage of washing the activated carbon with water, at a temperature of from 20 to 150° C., preferably from 40 to 100° C., followed by a stage of thermal treatment of the activated carbon thus washed, at a temperature of from 450 to 1000° C. approximately. It is thus possible by said treatment to remove partially or totally the compounds retained by the activated carbon and to re-use the activated carbon in a new contacting with an ethanolamine in order to improve the quality of the colour of the ethanolamine.

One of the advantages of the process according to the invention is not only the supplying of an ethanolamine of improved colour quality and in particular a colourless ethanolamine, as defined above, but also an ethanolamine whose improved colour quality is far more resistant over time and which in particular remains colourless for a long storage period.

Another advantage of the present process is the supplying of an ethanolamine whose content by weight of metal and in particular of iron is particularly reduced and becomes extremely low, for example less than 6 parts by weight per million (ppm), preferably equal to or less than 5 ppm or even less than 4 ppm.

The process of the invention can be used during or after the stage of preparation of the ethanolamine, more particularly during or after the stage of synthesis of the ethanolamine performed by reacting ethylene oxide with ammonia, preferably in aqueous medium. The process of the invention can be carried out preferably during or after the stage of purification of the ethanolamine, more particularly performed by one or preferably more than one distillation. In particular, the process of the invention can be used after having separated and isolated a crude ethanolamine from water and/or from one of the reagents in excess or not having reacted during the preparation of the ethanolamine, such as ammonia. It can be used more particularly during or after the stage of purification of the crude ethanolamine freed of water and optionally one of the reagents in excess and/or not having reacted, more particularly by one or more distillations, so as to separate and isolate substantially the MEA, the DEA and the TEA or a mixture of the latter. For example, the process of the invention can be used after the purification stage intended to separate and isolate the MEA and before that intended to separate and to isolate the DEA and the TEA or a mixture of TEA and DEA. The process of the invention can also be used after the purification stage intended to separate and to isolate the DEA and before that intended to separate and to isolate the TEA or a mixture of TEA and DEA. The process of the invention can preferably be used after or at the end of the stage of purification of the ethanolamine, more particularly after or at the end of the stage of purification of the TEA or a mixture of TEA and DEA, in particular at the moment when the TEA or a mixture of TEA and DEA is, isolated or more particularly stored in a storage zone, more particularly at a temperature extending from ambient temperature (about 20° C.) to 80° C., preferably from 25 to 70° C., more particularly from 30 to 60° C.

The present invention also relates to a process for manufacturing a triethanolamine (TEA) having an improved colour quality, which process comprises the following stages:
(i) a stage of synthesis of the TEA by contacting ammonia with ethylene oxide in aqueous medium so as to form a crude TEA comprising monoethanolamine (MEA), diethanolamine (DEA) and (TEA), as a mixture with water and ammonia in excess and/or not having reacted,
(ii) a stage of separation of the crude TEA and the mixture of water and ammonia so as to isolate and to recover the crude TEA, and
(iii) a stage of purification of the TEA by distillation of the crude TEA to as to separate substantially the MEA and the DEA from the TEA and to isolate and recover a purified TEA containing at least 85% by weight of TEA,
   which process is characterised in that, after the separation stage (ii) or during or after the purification stage (iii), the crude or purified TEA is contacted with an activated carbon free of one ore more metals chosen from rhenium, ruthenium, rhodium, palladium, osmium, iridium, platinum and silver.

The process of the invention is preferably used continuously, so that the stages described above and the contacting of the crude or purified TEA with the activated carbon are carried out continuously. The separation stage (ii) and the purification stage (iii) are preferably performed by one or more distillations, more particularly in series, and in particular continuously.

In the process for manufacturing TEA, the contacting of the crude or purified TEA with the activated carbon can be carried out according to one of the preferred variants described above in the process for preparing an ethanolamine of improved colour quality. The crude or purified TEA can be initially coloured, prior to its contacting with the activated carbon, and have a colour index (according to ASTM standard D 1209) of more than 40 Pt/Co, preferably more than 50 Pt/Co, and optionally a content by weight of metal, more particularly of iron, of more than 6 ppm, preferably more than 8 or 10 ppm. The method of analysis for measuring the iron content of the TEA can be that described above, namely plasma emission spectrometry (ICP-AES).

In the process for manufacturing TEA according to the invention, the contacting of the crude or purified TEA with the activated carbon can be carried out for a period sufficient to reduce the colour of the TEA, preferably a period such that the TEA becomes colourless, or more particularly a period such that the colour index (according to ASTM standard D 1209) becomes equal to or less than 50 or 40 Pt/Co, preferably equal to or less than 30 Pt/Co, more particularly equal to or less than 25 Pt/Co. The mean residence time of the crude or purified TEA contacted with the activated carbon can be chosen in a range of from 10 minutes to 18 hours, preferably from 30 minutes to 12 hours, in particular from 1 to 8 hours.

In the process for manufacturing TEA, the contacting with the activated carbon can be carried out at the earliest after stage (ii) for separating the crude TEA, that is to say once the crude TEA has been separated from the water and the ammonia, and in particular prior to stage (iii) for purification of the TEA. It can be carried out on the crude TEA substantially free of water and ammonia, and in particular prior to stage (iii) for purification of the crude TEA.

The contacting with the activated carbon can also be carried out during stage (iii) for purification of the TEA, that is to say at any moment of the purification of the TEA. In particular, it can be carried out after the moment when the MEA is first of all substantially separated from the crude TEA, and before the moment when the DEA is substantially separated from the TEA. It can in particular be carried out after the moment when the DEA is substantially separated from the TEA, and in particular before the moment when the TEA is purified of said latter impurities.

The contacting with the activated carbon can preferably be carried out after or at the end of stage (iii) for purification of the TEA, and consequently on the purified TEA containing less than 15%, preferably less than 10%, more particularly less than 1% or even less than 0.2% by weight of DEA. It can be carried out at the end of stage (iii) for purification of the TEA, before, during or after the purified TEA has been cooled to a temperature ranging from ambient temperature (about 20° C.) to 80° C., preferably from 25 to 70° C., in particular from 30 to 60° C., or else after the recovery of the purified and cooled TEA, for example in a zone for storing the latter, more particularly at a temperature ranging from ambient temperature (about 20° C.) to 80° C., preferably from 25 to 70° C., in particular from 30 to 60° C. The contacting with the activated carbon can be carried out in the zone for storing the purified TEA, which is more particularly taken from the storage zone so as to form a stream of purified TEA which then passes through a fixed bed of activated carbon and which can be finally returned in part or in toto to the storage zone. Any other, non-returned part can be isolated in order to be sent to the customers.

One of the advantages of the process for manufacturing TEA is the supplying of a TEA of improved colour quality and in particular a colourless TEA as defined above, and also a TEA whose improved colour quality is far more resistant over time and which in particular remains colourless for a long storage period.

Another advantage of the present process for manufacturing TEA is the supplying of a TEA whose content by weight of metal and more particularly of iron is particularly reduced and becomes extremely low, for example less than 6 ppm, preferably equal to or less than 5 ppm or even equal to or less than 4 ppm.

The following examples illustrate the present invention.

### EXAMPLE 1

A purified and coloured TEA was manufactured continuously according to the following process. The process comprises a stage (i) for the synthesis of TEA by reacting ammonia with ethylene oxide in aqueous medium, in a molar ratio between ammonia and ethylene oxide of 2.1/1, at a temperature of 60° C. A mixture of a crude TEA with water and ammonia not having reacted is thus obtained. The mixture is then subjected continuously to a stage (ii) for separation of the crude TEA, carried out by distillation in two distillation columns arranged in series, so as to separate first water and then ammonia from the crude TEA. A crude TEA freed of water and ammonia is thus obtained, which is then subjected continuously to a stage (iii) for purification of the TEA, carried out by distillation of the crude TEA in three distillation columns arranged in series, so as to separate successively the MEA and then the DEA and the TEAE substantially from the TEA, and to form a purified and coloured TEA. A sample of said purified and coloured TEA is taken: it has a content of TEA of more than 99 wt %, contains 6 ppm of iron (according to the plasma emission spectrometry method (ICP-AES) described above) and has a colour index (according to ASTM standard D 1209) of 70 Pt/Co.

The TEA sample is treated according to the following process. At ambient temperature (20° C.), the TEA sample is taken according to a continuous stream at a flow rate of about 1 ml/min. The TEA stream is sent through a fixed bed of 46 g of an activated carbon known under the commercial reference “SGL 8×30”® and sold by Chemviron Carbon (France); The activated carbon is free of metals chosen from rhenium, ruthenium, rhodium, palladium, osmium, iridium, platinum and silver. It has a bulk density of 0.46 g/cm³ and is in the form of particles having a grain size distribution such that less than, 15% by weight of the particles have a size equal to or more than 2.36 mm and less than 4% by weight of the particles have a size less than 0.6 mm. The activated carbon has a specific surface area (according to the N₂BET method described by Brunauer, Emmett and Teller in J. Am. Chem. Soc. 60, 309 (1938)) of 1000 m²g, and has a hardness index/number of 95 N. The mean residence time of the TEA in contact with the activated carbon is about 2 hours. A purified TEA of improved colour quality is thus recovered continuously, having a purity of more than 99% by weight, a colour index (according to ASTM standard D 1209) of 30 Pt/Co and an iron content of 4 ppm (according to the plasma emission spectrometry method (ICP-AES) described above).

### EXAMPLE 2

Exactly the same procedure is adopted as in Example 1, except that in the last distillation a purified and coloured TEA is recovered containing 91 wt % of TEA, 3 wt % of DEA, 5 wt % of TEAE and 8 ppm of iron (according to the plasma emission spectrometry method (ICP-AES) described above) and having a colour index (according to ASTM standard D 1209) of 120 Pt/Co and that a sample of said TEA is thus isolated.

The sample is treated as in Example 1, except that the sample prepared in Example 2 is used. A purified TEA is thus obtained continuously, having an improved colour quality, exhibiting in particular a colour index (according to ASTM D 1209) of 50 Pt/Co, and having an iron content of 5 ppm (according to the plasma emission spectrometry method (ICP-AES) described above).

1-10. (canceled)

11. Process for preparing an ethanolamine having an improved colour quality, characterised in that it comprises a contacting of an ethanolamine with an activated carbon free of one or more metals chosen from rhenium, ruthenium, rhodium, palladium, osmium, iridium, platinum and silver, under an atmosphere free of hydrogen.

12. Process according to claim 11, characterised in that the ethanolamine is an ethanolamine or a mixture of two or more ethanolamines chosen from monoethanolamine (MEA), diethanolamine (DEA) and preferably triethanolamine (TEA).

13. Process according to claim 11, characterised in that the ethanolamine is prepared in a synthesis stage by reacting ethylene oxide with ammonia, preferably in aqueous medium.

14. Process according to claim 11, characterised in that the ethanolamine has initially, prior to its contacting with the activated carbon, a colour index (according to ASTM standard D 1209) of more than 40 Pt/Co, preferably more than 50 Pt/Co, and optionally a content by weight of metal, preferably of iron, equal to or more than 6 parts per million (ppm), more particularly equal to or more than 8 ppm, in particular equal to or more than 10 ppm.

15. Process according to claim 11, characterised in that the activated carbon has a specific surface area (N₂ BET) of from 500 to 5000 m²/g, preferably from 500 to 2500 m²/g, more particularly from 700 to 2000 m²/g.

16. Process according to claim 11, characterised in that the contacting of the ethanolamine with the activated carbon is carried out at a temperature of from 10 to 200° C., preferably from 15 to 100° C., more particularly from 20 to 80° C.

17. Process according to claim 11, characterised in that the contacting of the ethanolamine with the activated carbon is carried out for a period sufficient to reduce the colour of the ethanolamine, preferably for a period such that the colour index (according to ASTM standard D 1209) of the ethanolamine becomes equal to or less than 50 Pt/Co, preferably equal to or less than 40 Pt/Co, more particularly equal to or less than 30 Pt/Co.

18. Process according to claim 11, characterised in that the mean residence time of the ethanolamine contacted with the activated carbon is chosen in a range of from 10 minutes to 18 hours, preferably from 30 minutes to 12 hours, more particularly from 1 to 8 hours.

19. Process according to claim 11, characterised in that it is carried out during or after the stage of preparation of the ethanolamine, preferably during or after the stage of purification of the ethylene.

20. Process for manufacturing a triethanolamine (TEA) having an improved colour quality, which process comprises the following stages:
(i) a stage for synthesising TEA by the contacting of ethylene oxide with ammonia in aqueous medium, so as to form a crude TEA containing monoethanolamine (MEA), diethanolamine (DEA) and TEA, as a mixture with water and ammonia in excess and/or not having reacted,
(ii) a stage for separating the crude TEA and the mixture of water and ammonia, so as to isolate and recover the crude TEA, and
(iii) a stage for purifying the TEA by distillation of the crude TEA, so as to separate substantially the MEA and the DEA from the TEA, and to isolate and recover a purified TEA containing at least 85 wt % of TEA,
which process is characterised in that, after the separation stage (ii) or during or after the purification stage (iii), the crude or purified TEA is contacted with an activated carbon free of one or more metals chosen from rhenium, ruthenium, rhodium, palladium, osmium, iridium, platinum and silver, under an atmosphere free of hydrogen.
